(19) United States
(12) Patent Application Publication
(10) Pub. No.: US 2020/0197351 A1
(43) Pub. Date: Jun. 25, 2020

(54) TRIVALENT AND DIVALENT CATIONS AS ADMINISTRABLE AGENTS FOR INCREASED PROCESSIVITY AND IMPROVED FIDELITY OF REVERSE TRANSCRIPTASE IN TELOMERASE AND IN NUCLEIC ACID POLYMERASES

(71) Applicants: Aaron Gershon Filler, Santa Monica, CA (US); Andrew ML Lever, Cambridge (GB)

(72) Inventors: Aaron Gershon Filler, Santa Monica, CA (US); Andrew ML Lever, Cambridge (GB)

(21) Appl. No.: 16/589,042

(22) Filed: Sep. 30, 2019

Related U.S. Application Data
(60) Provisional application No. 62/739,347, filed on Oct. 1, 2018.

(51) Int. Cl. A61K 31/28; A23L 33/165

(52) U.S. Cl. CPC A61K 31/28; A23L 33/165

(57) ABSTRACT

A novel method of increasing longevity by improving the processivity, fidelity, and activity of the telomerase enzyme is provided by agents which introduce trivalent cations such as scandium 3+ into the nuclei of cells, whereupon there occurs substitution of the trivalent cation into the place of the natural divalent cations such as magnesium or calcium or manganese. This substitution optimizes the adherence of the telomerase enzyme to its nucleic acid template with the result that cellular replicative senescence is delayed and deferred in various cell types. The trivalent cations can be introduced topically, orally as a food additive or supplement, or as a medication for oral, intramuscular, intrathecal or intravenous administration.

### A. CROSS REFERENCE TO RELATED APPLICATIONS

This application claims the benefits including priority under 35 U.S.C. § 119(e) to the following prior filed U.S. Provisional Application: Ser. No. 62/739,347 filed Oct. 1, 2018, as it relates to this definitive non-provisional application to be filed by Sep. 30, 2019 in compliance with 37 C.F.R. 1.78 (a)(1-4), the contents of which, together with any attachments submitted with it, are incorporated in this disclosure by reference in their entirety.

### B. FIELD OF THE INVENTION

The present invention relates generally to the field of food additives or supplements, topically administered agents, orally ingested agents/medicaments and medications for topical, oral, intramuscular, intrathecal or intravenous applications. More specifically, it is in the field of administered agents intended to affect the progression of aging and longevity in mammals including humans.

The subfield is of agents intended to manipulate the function of the natural telomerase enzyme(s) present in the cells of mammals including humans. Further subfields are the use of an introduced agent to alter and thereby improve or increase the processivity of telomerase enzymes—which is to positively affect, increase and accelerate the rate of progression and the activity level of said enzymes in the task of repairing the telomere portion at the ends of the chromosomes of mammals and humans; and to alter and improve the fidelity of telomerase enzymes—which is to affect and improve the accuracy of correctly copying and restoring the DNA of the telomeres of mammals including humans; and to alter and increase or improve the number and variety of situations wherein the telomerase enzymes are subject to activation in the cells of mammals including humans which is to assure that there is an increase in the variety of replicating cells in the body of mammals including humans in which telomerase is effectively active to preserve or restore the length and utility of telomeres and to thereby extend or increase the number of replications each cell type can engage in.

In all of these subfields, the purpose of the invention is to prevent or reduce the breakdown of telomeres so as to prevent or defer the onset of cellular senescence or termination of cellular replication due to aging with the overall objective of improving the health and longevity of cells in mammals including humans and so to improve the health and longevity of the individual mammal including humans.

### C. BACKGROUND OF THE INVENTION

### 1. Telomeres, Cellular Senescence and Aging

In all eurkaryote life (plants, animals and fungi) chromosomes exist that face a problem with repeated replication. In prokaryotes—such as bacteria—there is typically a single circular chromosome. In eukaryotes, there is a nucleus in the cell, and it contains linear chromosomes. The enzyme that replicates the chromosomal DNA during cell division—DNA polymerase—doesn't work properly on the end of a linear chromosome. Therefore, with each replication, parts of the end of a chromosome are not replicated and the end shortens, losing genetic information. If the process is left unchecked, with each replication of the cell, chromosomal information is destroyed until a crisis is reached that leads to the senescence and death of the cell and its entire lineage of descendants—which all arrive at the crisis point at the same time.

This molecular clock appears to be among the most fundamental bases for the loss of vitality with aging.

In eurkaryotes, there is a protective mechanism available that can prevent replicative senescence which centers around a structure called a telomere. This is an end-cap on the end of each linear chromosome strand. The telomere is made of DNA that doesn't encode any useful product. It is there to be whittled away with each round of replication so that the losses will not destroy any critical genes. However, the telomere is of fixed starting length in a newly born individual. Therefore, in about 70 cycles of replication, the telomere gets used up, and chromosomal destruction commences, leading to cellular senescence and death by aging.

The telomere replicative consumption process can be prevented by a natural enzyme called telomerase. In certain cells in the human body, for instance, a high level of telomerase function repetitively restores the telomeres to a full healthy length. This occurs in lymphocytes—which must undergo many rounds of replication to supply the body with a constant, sufficient, and dynamic immune response, and it occurs in germ-line cells—that form sperm, and it occurs in some stem cell lines maintained in the body.

Some cells—such as muscle cells and neurons in the brain—are “post-mitotic” in that once the appropriate number of them is reached during embryogenesis and growth—they never divide. Therefore, for these post-mitotic cells, their telomere length has no effect and they do not suffer from cellular senescence.

However, for some tissues—such is in the skin, the lining of gut, and in a variety of locations around the body—it is necessary to provide a steady stream of replacement cells. These cells, absent sufficient support from telomerase can manage about 70 rounds of replication and then become 1) unable to replicate and 2) subject to crisis and death (apoptosis) because one too many replicative attempts, commences chromosomal destruction. Once the telomere is used up, raw ends of chromosomes trigger a complex cascade of destruction by enzymes, and fusion with other raw chromosome ends that is lethal and irreversible.

The telomere consumption process has a benefit, however. If a cell suffers a mutation that makes it cancerous, it starts to replicate at a high rate. However, after 60 or 70 doublings, the telomeres are destroyed and the cancer self-annihilates. In tumors that survive this crisis and proceed to attack the rest of the body, the cells have typically reactivated their telomerase—getting restoration of fresh full length chromosome telomeres with each cancerous replication—or they rely on an “alternative lengthening of telomeres” (ALT) process that can preserve telomere length.

One other issue in telomerase function is that errors in telomerase function can lead to premature telomere failure.

### 2. General Efforts to Investigate Telomerase

Given this background information—detailed in as many as 20,000 peer reviewed research publications—it is apparent that innovation directed at prolonging human life would be well directed at discovering a way to nudge the telomerase system into more prolonged and widespread productive use, without unleashing unrestrained tumor growth.

One of the great unsolved paradoxes of medicine and health is the bivalent status of telomerase. Telomerase enzymes allow death by aging in 67% of the population when they function at too low a level to prevent chromosomal senescence, yet an increased activity of telomerase is implicated in carcinogenesis that causes death in another 23%. Once these two opposed but telomerase associated causes of death are accounted for, there are just 11% of the population that die from trauma, infections, intoxications, hunger and other causes.

It has been axiomatic for 20 years that if telomerase activity in cells could be increased then aging effects in many tissues could be mitigated. Yet far more resources have been devoted to inhibiting and suppressing telomerase as a means of trying to treat cancers.

It has seemed impossible or unlikely that any single strategy or type of food supplement or medication could upregulate telomerase in healthy cells without the risk of promoting cancer. Geron corporation, for instance, devoted tremendous energies, prosecuted hundreds of patents, and attracted large sums of venture capital before finally abandoning any hope of affecting aging after nearly two decades of intensive effort. Undaunted, Google has founded Calico to look elsewhere than telomerase for a new line of attack on senescence.

That is why there are 20,000 publications in the academic peer reviewed literature and thousands of patents as well that deal with telomerase. One generally needs only to read the first few lines to learn whether telomerase will be the enemy to be defeated or the reluctant champion to be encouraged. It is difficult to find any method that proposes a means of suppressing the negative effects and encouraging the positive effects with a single strategy.

### 3. General Efforts to Investigate Reverse Transcriptase

“reverse transcriptase” (RT)—it reads an RNA template to form a DNA copy. In nature and in medicine, the vast amount of interest in reverse transcriptase concerns certain classes of viruses such as the Human Immunodeficiency Virus (HIV) or the Avian Myeloblastosis Virus (AMV) which typify some disease causing processes. In those “retroviruses” the genome of the virus is stored in RNA instead of the DNA used in prokaryote bacteria, and eukaryotes. In HIV, for instance, the RT copies the virus's RNA genome into a DNA version after invading a human cell. It then gets the cell to use its human DNA polymerase enzyme molecules to make multiple copies of the invader DNA and then allows the cell to use its “RNA polymerase” to make RNA from the DNA. The RNA produced then gets assembled into thousands of new viruses that can attack other cells.

Because of this issue, a vast amount of research has been devoted to learning ways to inhibit or disable reverse transcriptase as a means of treating AIDS (acquired immune deficiency syndrome).

Promoting reverse transcriptase function is therefore problematic as a medical goal because it seems likely to increase growth rates for lethal viruses, or to excessively promote telomerase in such a way as to promote cancer.

Cation Substitution to Alter the Function of Reverse Transcriptase and Nucleic Acid Polymerase

In this invention, the inventors draw upon two entirely serendipitous discoveries that emerged from Mill contrast agent research more than twenty years ago that led to a process for manipulating the retroviral reverse transcriptase enzyme.

The method increased the speed and accuracy of retroviral reverse transcriptase. However, the same method is applicable to RNA polymerase and DNA polymerase although those types of enzymes have far more elaborate arrays of methods to check accuracy and correct copying errors.

A DNA or RNA polymerase or reverse transcriptase must adhere to a template with the purpose of making a copy of the template that preserves and duplicates the information content. For DNA polymerase and RNA polymerase the template is written out in DNA and for reverse transcriptase, the template is written out in RNA.

Processivity describes the ability to move from step to step—nucleotide to nucleotide—without falling off the template or stopping. There is also a measure of the process that may be described as the rate of catalysis, which describes how long the enzyme takes to catalyze each reaction cycle. Catalysis involves, at least, adhering to the template, recognizing which deoxyribose nucleotide DNA “letter” is presented—C G T A (cytosine guanine thymine adenine), capturing the correct complementary monomer (G C A T), attaching and bonding the monomer to the growing new chain, releasing the template monomer and moving to the next monomer. For RNA, the ribose nucleotides are similarly identified but for uracil (U) in place of the thymine. The time required for a nucleic acid polymerase to finish a complete copy of a given length of template will be determined by both the enzymatic rate of catalysis (how long it takes to perform each of the monomer steps just described) and by the processivity—the amount of time it can proceed repetitively before it falls off the template and has to re-attach before continuing on with its work.

In this invention, as to reverse transcriptase, it is possible to increase processivity at the same time that fidelity is increased. The rate of transcription can be increased by the same agents that reduce errors. Speed here is a result of enzymatic rate and processivity, while errors are failures of fidelity.

In many areas of science, technology, and even polymerase biology, the opposite is common. If a job is done ten times faster, there will be more errors. This is the situation where an information copying system is functioning at its highest effective speed and moving faster is only possible by allowing more incorrect reads—increased processivity correlated with decreased fidelity: an overdriven inefficient polymerase.

A second alternative situation is a polymerase that progresses with atypical slowness because it is inefficient and makes many errors—having difficulty incorporating monomers. In that situation, when compared to a different polymerase that has a higher rate of proceeding along a template intrinsically, we might expect that if the inefficient polymerase were driven to work more quickly then more errors of the slow and ineffective polymerase would result, but by switching to a different and more efficient polymerase there would be an improvement in the situation—increased processivity with increased fidelity: a different (or altered) polymerase that works more effectively.

Yet, with regard to the functioning of the reverse transcriptase of retroviruses such as the HIV virus and Avian myeloblastosis virus (AMV), the inventors have shown previously that increased processivity and increased fidelity can result from the same process. This is because of weak template adhesion in the recognition area of the reverse transcriptase. Here, the inventors consider the situation where there is only one polymerase available to be considered and we are assessing whether the speed of that polymerase can be increased, rather then comparing two different polymerases in the laboratory—increased processivity and increased fidelity—an inefficient polymerase that is made to work more effectively by altering its conditions of work.

To be useful in humans as to their telomerase function, it will not be possible to change or replace the polymerase enzyme. However, if it is routinely feasible to so change the working conditions as to make it easier for the polymerase to do its job in its usual setting, then the unaltered polymerase enzyme may get the job done faster with increased accuracy.

The speed of the work is important because there may be a very limited time during a mitotic event wherein a telomerase can work. It must be able to complete a repair/restoration in that time frame and to complete it with high fidelity.

### 4. Effects of Template Secondary Structure on Processivity

The inventors have shown previously that the processivity of reverse transcriptase can be increased by as much as ten times by the substitution of trivalent cations for the divalent cations normally required for template based extension. At the time of reporting on this phenomenon, the detailed mechanism was not known. However, subsequent laboratory work revealed that template adhesion is the relevant issue.

In these experiments, a template from the of RNA genome of the retrovirus was used. RNA templates can have complex three dimensional tertiary structure because of links between pairs of nucleotides in the unpaired strand. The result is a complex array of fingers and hairpin loops into which a given molecule of RNA forms on a strictly reproducible structural basis dependent on the nucleotide base sequence.

The experiments showed that as—for instance—the reverse transcriptase of AMV progressed along its template, it would fall off the template at predictable locations—typically at the sharp turn at the point of the hairpin loop.

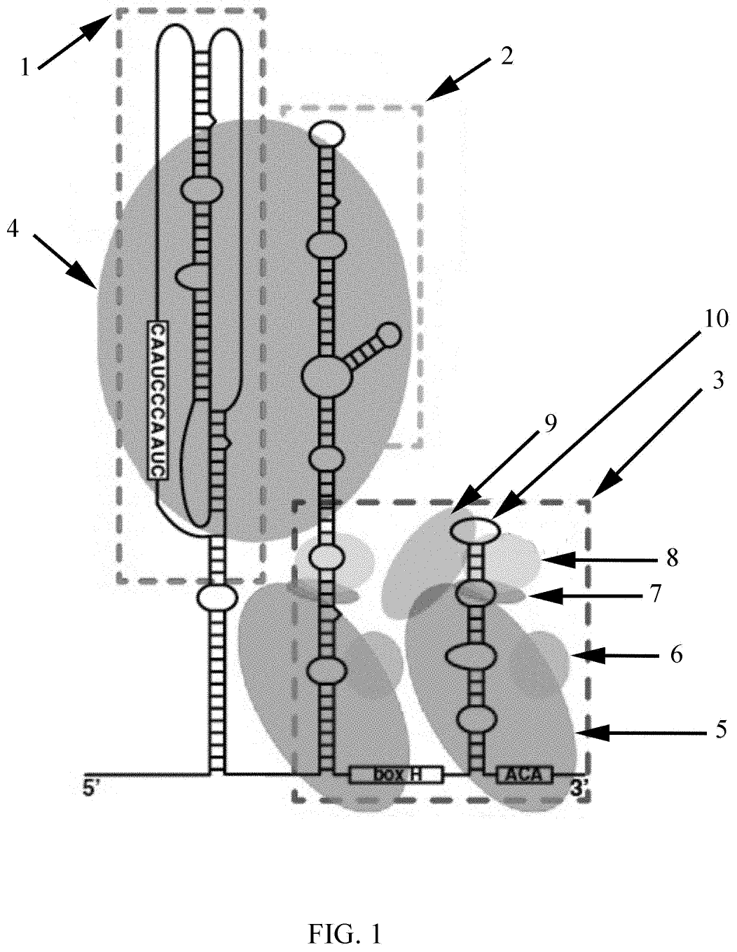

A depiction of the human telomerase RNA (hTR) is shown in FIG. 1: FIG. 1. Secondary structure and known protein components of the human telomerase holoenzyme. The human telomerase RNA (hTR) contains three major structural and functional domains: the core domain 1; the CR4/CR5 domain 2, and the H/ACA scaRNA domain 3. The hTR core and CR4/CR5 domains independently bind the hTERT 4. The hTR scaRNA domain binds two sets of the four H/ACA RNP proteins: dyskerin 5, Gar1 6, Nop10 7, and Nhp2 8, The protein TCAB1/WDR79 9 binds both the dyskerin and the CAB box located at the CR7 region 10 within the H/ACA scaRNA domain. Abbreviations: (RNA—Ribonucleic Acid); scaRNA (small Cajal body specific RNA); H/ACA (H box region and ACA box region); hTERT (human telomerase reverse transcriptase); RNP (Ribonucleoprotein); Nop10 (nucleolar protein 10); CAB box (Cajal body box); TCAB1 (telomere Cajal body protein 1). From Zhang et al., Architecture of Human Telomerase, 108 PROCEEDINGS OF THE NATIONAL ACADEMY OF SCIENCE 20325-20332 (2011).

### 5. Use of Substitution with Trivalent Cations to Improve Processivity

The inventors tested AMV reverse transcriptase on a template of AMV RNA genome. The process produces complete transcripts of the entire genome, but it also produces a large number of fragments of various length. When the reaction is halted, the fragments can be applied an electrophoresis type gel, and run out so that they are pulled down the length of the gel by electrostatic forces. The larger fragments encounter more resistance as they are dragged through the gel and travel more slowly, while the smaller fragments move more rapidly. The monomers used to support the reaction are radiolabelled with radioactive phosphorus.

The electric current driving the electrophoresis is turned off and a photographic film is exposed. The fragments create a pattern on the resulting image:

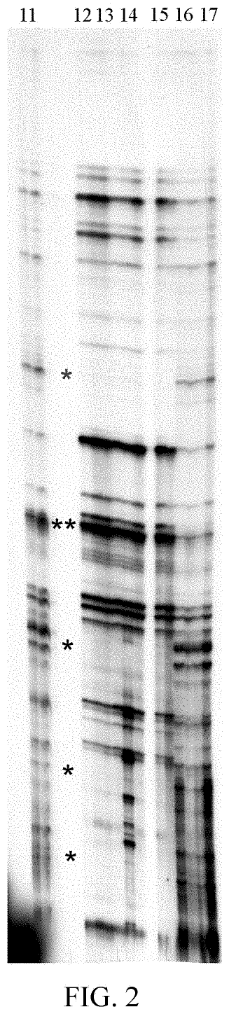

FIG. 2—AMV Transcript Analysis Gel. Transcript extension analysis of AMV reverse transcriptase using the Hind III fragment of the HIV-1 genome (bases 541-1086 Los Alamos data base numbers) as a physiologic template to assess effects of various cations with and without addition of magnesium in the setting of transcription with preserved RNA secondary structure. Template dependent lengths of cDNA transcripts show locations where the enzyme dissociates from template at points of complex secondary structure (e.g. hairpins, loops) resulting in standard sets of partial transcripts. Single asterisks (*) mark read-through events where the enzyme does not fall off the template when a higher ionic charge trivalent cofactor is substituted for magnesium in the recognition/adhesion site of the enzyme on the negatively charged template. This results in a larger relative amount of longer transcripts and demonstrates improvement of this aspect of processivity. Alteration in the shape of the template secondary structure due to direct effects of the cation on the template may also play a role. Double asterisk (**) marks a site where a significant increase of read-through occurs when both trivalent and divalent magnesium are supplied by comparison with enzyme behavior with magnesium alone. Magnesium channel 11, Lanthanum channel 12, Lutetium channel 13, Yttrium channel 14, Terbium channel 15, Terbium+Magnesium channel 16, Scandium+Magnesium channel 17.

In the laboratory, a reaction such as this requires that a solution be prepared that contains template, monomers, enzyme and a divalent cation such a Mn2+ (manganese) or Mg2+ (magnesium). What the gel depicts is that the reaction produces large amounts of fragments of specific lengths.

When the fragments are analyzed, they line up with the linear portions of the HIV-1 genome. Magnesium is the usual divalent cation co-factor and the channel on the left shows the typical fragments. These fragments result when the reverse transcriptase falls off the template. The analysis reveals that the enzyme falls off the template at the hairpin loop points. That is, it runs steadily along the “straight” portions of the template but falls off as it has to follow the template around a sharp curve. The enzyme then reattaches and proceeds on, producing additional fragments. Ultimately, the fragments are joined together in vivo and a complete copy is completed.

Notice however, in FIG. 2 that when a different cation is substituted for magnesium or added to it, a different pattern results.

The cation plays a role wherein one charge attracts the template and the second charge attracts the enzyme. It helps to hold the two together as the process of transcription proceeds.

If a different divalent cation of larger physical size but higher electropositivity is used, then no improvement results because the large size actually inhibits function, despite the stronger attraction, or the attraction is so great that the normal separation and translocation cannot occur.

However, when trivalent cations are used in place of or in addition to the Mg2+, then we see that the reverse transcriptase reads through some of the hairpin loops without falling off. The result is that some of the fragments disappear or are reduced in quantity.

### D. METHODOLOGY FOR PERFORMING FOR PROCESSIVITY ASSAY

### 1. Methods and Materials for Viral Genome Processivity Assay

### a. Materials

Poly rA, oligo dT, and Avian Myeloblastosis Virus (AMV) reverse transcriptase were purchased from Pharmacia, and ³²P dTTP was from Amersham. CeCl₃.7H₂O, GdCl₃.6H₂O, LuCl₃.6H₂O, ScCl₃.xH₂O, YCl₃.6H₂O, TbCl₃.6H₂O, RuCl₃.xH₂O were from Aldrich. AlCl₃.6H₂O, KCl, LaCl₃.7H₂O, LiCl, MgCl₂.6H₂O, MnCl₂.4H₂O, RNAse free bovine serum albumin (BSA), dithiothreitol (DTT), dTTP, HEPES base, Tris HCl, and Tris base were purchased from Sigma. Peroxide free NP-40 was from Boehringer Mannheim.

### b. Preliminary Cocktails

To limit the potential effects of precipitation and polymerization of the metal cations we prepared pre-reaction RT cocktails in 200 mM buffer with no metal cations added. The reactions are subsequently initiated by addition of various concentrations and mixtures of MxCl₃ and MgCl₂ (where Mx is the metal under study) prepared in advance as stable solutions in 0.1N HCl.

Experiments were conducted in 150 mM Tris pH 8.2 which (excluding cations) were made in deionized, metal free water with 75 mM KCl, poly-rA 5 μg/ml, oligo dT₍₁₂₋₁₈₎ 5 μg/ml, and [a³²P]dTTP 5 μCi/ml (spec. act. 400 Ci/mmol). In all cases the cocktails were prepared in advance then transferred to 96 well plates in volumes of 100 μl/well, and the plates then frozen.

### c. Metal Cation Preparation

Double distilled water was passed through Sigma C-7901 chelating resin and used to prepare 0.1N HCl for dissolving the various metal chlorides to prepare 250 mM metal solutions. Serial dilutions of all the cation solutions were then made in 0.1N HCl to set up final stable solutions prepared as 75 mM metal chloride and 7.5 mM metal chloride. These were then used to prepare 96 well ‘feeder’ plates containing the various metals to generate the final assay cocktails in the various final concentrations.

For each metal cation assay, there were at least eight cation conditions set up for each experimental channel. These included four concentrations of only the metal cation being evaluated: 0.1 mM, 0.5 mM, 1.0 mM, 3.0 mM, and four mixtures with magnesium, 1.0 mM MxCl₃+0.5 mM MgCl₂, 1.0 mM MxCl₃+1.0 mM MgCl₂, MgCl₂, 1.0 mM MxCl₃+3.0 mM MgCl₂ and 3.0 mM MxCl₃+3.0 mM MgCl₂.

Because of precipitation of lanthanide metals at concentrations greater than 1.0 mM in these cocktails, a further set of experiments with lanthanum and scandium cations were conducted in the presence of 3 mM Nitrilotriacetic acid (NTA), ethylene diamine tetraacetic acid (EDTA), or Diethylene triamine pentaacetic acid (DTPA) with and without Magnesium as follows: 0.1 mM, 0.5 mM, 1.0 mM, 3.0 mM, 1.0 mM MxCl₃+0.5 mM MgCl₂, 1.0 mM MxCl₃+1.0 mM MgCl₂, MgCl₂, 1.0 mM MxCl₃+3.0 mM MgCl₂ and 3.0 mM MxCl₃+3.0 mM MgCl₂. The chelate solutions were made up in advance with stoichiometric amounts of meglumine per acetic acid in the various chelators to correct their pH to be close to that of the buffer. Independent controls were also run to assure that the meglumine itself had no effect on the reactions.

### d. RT Assay Reactions

Assays with AMV involved 5 μl of purified enzyme (1 unit/μl) added to 40 μl of thawed cocktail. Reactions were started by transferring the various metal cation dilutions and MgCl₂ dilutions as 5 μl volumes to trays with the full cocktails. All experiments included three replications with timed row by row application of the activating metal solution with eight or twelve channel multi-pipettors. Lanthanide and control assays ran at 37° C. for 90 minutes.

### e. RT Assay Analysis

Reactions were terminated by dot transfers of 5 μl to pre-numbered locations on 96 spot DE81 Whatman paper sheets which were then dried, washedⁱ in 2× SSC, and loaded into a Canberra Packard 96-matrix ß-counterⁱⁱ.

### f. Transcript Analysis

The AMV transcripts and gel experiments were conducted essentially as described in Harrison et al, The human immunodeficiency virus type 1 packaging signal and major splice donor region have a conserved stable secondary structure. 66 Journal of Virology 4144-4153 (1992); Harrison et al, Pausing of reverse transcriptase on retroviral RNA templates is influenced by secondary structures both 5′ and 3′ of the catalytic site, 26 Nucleic Acids Research 3433-3442 (1998), Filler et al, Effects of cation substitutions on reverse transcriptase and on human immunodeficiency virus production. 13 AIDS Research and Human Retroviruses 291-299 (1997), Filler AG, Lever AMLL. Nucleic Acid Amplification Using Scandium and Lanthanum Ions. U.S. Pat. No. 5,554,498 (1996), Filler AG, Lever AMLL. Particulates for Anti-Viral Therapy. U.S. Pat. No. 5,614,652 (1997). See also Potts, B. J. in Techniques in HIV Research (eds. Aldovini, A. & Walker, B. D.) 103-106 (Stockton Press, New York, 1990) and Filler AG. Reverse transcriptase microassay. Matrix Application Notes, Packard Instrument Company. PAN0035/MAN-016:1-8 (1993). Several Group IIIB and lanthanide cations were tested both with and without magnesium.

The Hind III fragment of HIV-1 strain IIIBⁱⁱⁱ from bases 541-1086 (Los Alamos AIDS data base numbering) was cloned into the HIND III site of Bluescript KS II (Stratagene) and RNA was transcribed in vitro from the T3 promoter. In vitro-transcribed RNA was extracted twice with phenol-chloroform and precipitated by addition of 0.1 volume of 3M sodium acetate pH 6.0 and 2.5 volumes of ethanol. The RNA was dissolved in TE and was incubated with 1 μg of RNase-free DNase I per 100 μg se 2 buffer for 30 min. RNA was then extracted twice with phenol-chloroform and precipitated by the addition of 0.1 volume of 3M sodium acetate pH 6.0 and 2.5 volumes of ethanol. A synthetic oligonucleotides (1 μl of 100 mM (primer)) was annealed to 1 μg of RNA in 10 μl of annealing buffer (40 mM Tris HCl (pH 8.3), 240 mM KCl, 4 mM dithiothreitol) by heating to 75° C. for 3 minutes and then allowing to cool over 15 min to room temperature in 200 ml of water.

Extension analyses from these primers were carried out with 1 unit of avian myeloblastosis virus reverse transcriptase (Northumbrian Biologicals Ltd, Cramlington, England) per 5 μl of hybridization mix at 42° C. for 30 min. in a solution containing 50 mM Tris HCl (pH 8.3), 40 mM KCl with 1 mM each dCTP, dTTP, and dGTP, and 1 μl of [a³²P]dATP (ICN-Flow, 3000 Ci/mM) with the addition of different cations, (with or without magnesium). cDNAs were precipitated under ethanol, washed with 70% ethanol dried and dissolved in TE, and 1/10 formamide dye mix was added. The samples were heated to 90° for 5 minutes before loading onto a 6% polyacrylamide-7M urea gel.

### 2. Methods and Materials for Telomerase Processivity Assay

Method as set forth in Chen et al, A single nucleotide incorporation step limits human telomerase repeat addition activity. 37 EMBO J., e97953, 1-17 (2018).

### a. In Vitro Reconstitution of TF Human Telomerase

Human TERT protein is expressed in rabbit reticulocyte lysate (RRL) from the pNFLAG-hTERT plasmid DNA using the TnT T7 Quick Coupled transcription/translation kit (Promega) following manufacturer's instructions (Xie et al, A novel motif in telomerase reverse transcriptase regulates telomere repeat addition rate and processivity, 38 Nucleic Acids Res 1982-1996 (2010). The hTR pseudoknot (residues 64-184) and CR4/5 (residues 239-328) fragments are in vitro-transcribed, gel-purified, and assembled together with the TERT protein in RRL for 30 min at 30° C. at a final concentration of 1.0 μM (Qi et al, RNA/DNA hybrid binding affinity determines telomerase template-translocation efficiency. EMBO J 31: 150-161 (2012); Brown et al, A self-regulating template in human telomerase. 111 Proc Natl Acad Sci USA, 11311-11316 (2014).

### b. In Vivo Reconstitution of Wild-Type Human Telomerase

HEK 293FT cells are grown in DMEM medium (Corning) supplemented with 10% FBS (Atlanta Biological), 1× Penicillin—Streptomycin-Amphotericin B mix (Lonza) and 5% CO₂ at 37° C. to 80-90% confluency. Cells in a 6-well plate are transfected with 0.4 μg of pcDNA-NFLAG-hTERT, 1.6 μg of pBS-U1-hTR wild type or template mutants, and 6 μl of FuGENE HD transfection reagent (Promega) following manufacturer's instruction. Cells are harvested 48 h post-transfection, homogenized in HEPES lysis buffer (20 mM HEPES-KOH, pH 7.9, 400 mM NaCl, 0.2 mM EGTA, 2 mM MgCl₂, 10% glycerol, 5 mM ß-mercaptoethanol, and 1× complete protease inhibitor cocktail (Roche), 1 mM PMSF), incubated on ice for 30 min and the lysate clarified by centrifugation. Two hundred microliters of cell lysate are combined with 30 μl Anti-FLAG® M2 Beads (Sigma, Cat # A2220) pre-washed with 1×TBS buffer (50 mM Tris-HCl, pH 7.4 and 150 mM NaCl) and incubated at 4° C. with gentle rotation for 1 h. The beads are washed three times with 100 μl of 1×TBS buffer and once with 50 μl 1× telomerase reaction buffer (50 mM Tris-HCl, pH 7.5, 3 mM MgCl₂, 50 mM KCl, 2 mM DTT, and 1 mM spermidine), followed by activity assay.

### c. & Measurement Using TF Telomerase

One microliter of RRL reconstituted TF telomerase enzyme is assayed in a 10 μl reaction containing 1× telomerase reaction buffer, 40 μM pre-annealed DNA/RNA duplex, specified dNTPs, and 0.165 μM of the denoted α-³²P-dNTP. For measuring the K_{M} values, the activity assays are performed with nucleotide concentrations varying from 0 to 200 μM, or up to 1 mM for high K_{M} measurement. Reactions are incubated at 30° C. for 60 min and terminated by phenol/chloroform extraction, followed by ethanol precipitation. The DNA products are resolved on a 15% (wt/vol) polyacrylamide/8M urea denaturing gel, dried, exposed to a phosphorstorage screen, and imaged on a Bio-Rad FX-Pro phosphorimager. The intensities of specific products are normalized to the total product intensity and plotted against the nucleotide concentrations with the Michaelis-Menten equation, Y=Vmax*X/(K_{M}+X), used to fit the nonlinear curve to determine the K_{M} (Prism 5, GraphPad Software).

### d. Activity Assay for dNDP and dNMP Incorporation

One microliter of RRL reconstituted telomerase enzyme, 1 unit of AMV RT (Promega), 0.5 units of Taq DNA pol III (NEB), 1 unit of T4 DNA pol (Fermentas), or 0.5 units of Klenow fragment of DNA pol I (Invitrogen) are assayed in 10 μl reactions containing 1× telomerase reaction buffer, 40 μM of denoted pre-annealed DNA/RNA or DNA/DNA hybrid substrates, 100 μM dGTP, dGDP, or dGMP, and 0.165 μM α-³²P-dATP. The assay with TGIRT III group II intron RT (InGex) contained 50 units of enzyme, 1× reaction buffer (20 mM Tris-HCl, pH 7.5, 5 mM MgCl₂, 0.45 M NaCl, 5 mM DTT) and 10 μM dGTP, dGDP, or dGMP, and 0.165 μM α-³²P-dATP. Reactions are incubated at 30° C. for 60 min and terminated by phenol/chloroform extraction, followed by ethanol precipitation. The size marker is prepared in a 10 μl reaction containing 1× reaction buffer (100 mM sodium cacodylate, pH 6.8, 1 mM CoCl₂, and 0.1 mM DTT), 1 μM oligonucleotide as indicated, 10 units of terminal deoxynucleotidyl transferase (TdT, Affymetrix), and 0.165 μM α-³²P-dATP (3,000 Ci/mmol, 10 mCi/ml; PerkinElmer). The reaction is incubated at 30° C. for 5 s and terminated by addition of 10 μl 2× formamide loading buffer [10 mM Tris-HCl, pH 8.0, 80% (vol/vol) formamide, 2 mM EDTA, 0.08% bromophenol blue, and 0.08% xylene cyanol]. The DNA products are resolved on a 15% (wt/vol) polyacrylamide/8 M urea denaturing gel, dried, exposed to a phosphorstorage screen and imaged on a Bio-Rad FX-Pro phosphorimager.

### e. Telomerase Direct Primer Extension Assay

Twenty microliters of immuno-purified in vivo-reconstituted telomerase enzyme on beads are assayed in a 10 μl reaction containing 1× telomerase reaction buffer, 1 μM DNA primer, specified dNTPs, and 0.165 μM of the denoted α-³²P-dNTP. Reactions are incubated at 30° C. for 60 min and terminated by phenol/chloroform extraction, followed by ethanol precipitation. The DNA products are resolved on a 10% (wt/vol) polyacrylamide/8 M urea denaturing gel, dried, exposed to a phosphorstorage screen, and imaged on a Bio-Rad FX-Pro phosphorimager. Repeat addition efficiency is estimated as the ratio of the high M.W. DNA products (>6 repeats added) over the low M.W. DNA products (1-6 repeats added). The cutoff at 6 repeats is arbitrarily chosen to divide the gel into approximately two even sections. The relative high/low ratios of reactions with varying nucleotide concentrations is determined through normalization to the ratio from the low nucleotide concentration reaction.

### f. Telomerase Product Release Assay for Repeat Addition Processivity Determination

Twenty microliters of immuno-purified in vivo-reconstituted telomerase enzyme on beads are assayed in a 10 μl reaction containing 1× telomerase reaction buffer, 1 μM DNA primer, a range of dNTPs, and 0.165 μM of the denoted α-³²P-dNTP. Reactions are incubated at 30° C. for 60 min, and the DNA products in the supernatant are separated from the DNA products bound to the telomerase enzyme immobilized on the beads. Following ethanol precipitation, the DNA products are resolved on a 10% (wt/vol) polyacrylamide/8 M urea denaturing gel, dried, exposed to a phosphorstorage screen, and imaged on a Bio-Rad FX-Pro phosphorimager. Repeat addition processivity is calculated using the equation: Processivity=−ln2/2.303κ (Latrick & Cech, POT1-TPP1 enhances telomerase processivity by slowing primer dissociation and aiding translocation. 29 EMBO J 924-933 (2010)). The slope, κ, is determined by plotting the intensity of each major band, normalized to the intensity of the first band, over the repeat number.

### g. Pulse-Chase Time Course Assay

Twenty microliters of immuno-purified in vivo-reconstituted telomerase enzyme on beads are initially pulsed with 0.165 μM α-³²P-dTTP for 5 min and then chased with 100 μM dTTP and denoted concentrations of dATP and dGTP. Aliquots of the reactions are terminated by phenol/chloroform extraction at denoted time points, followed by ethanol precipitation. The DNA products are resolved on a 10 (wt/vol) polyacrylamide/8 M urea denaturing gel, dried, exposed to a phosphorstorage screen, and imaged on a Bio-Rad FX-Pro phosphorimager. To determine the rate of repeat addition, the longest DNA products with the highest intensity above initial pulse product bands are used to deduce a “modal band” to calculate the extension rate as previously described (Drosopoulos et al, Human telomerase RNA template sequence is a determinant of telomere repeat extension rate. 280 J Biol Chem 32801-32810 (2005)). The modal bands are determined from the intensity traces generated by the ImageJ (NIH) program, with the number of repeats comprising the modal band plotted against the time point of the chase reaction, and the slope of the best-fit trend line determined the rate of repeat addition for the reaction.

### E. Agents for Administration

Scandium is a small trivalent cation that is present in the body in small amount. It is next to calcium on the periodic chart. However, it has little or no toxicity despite detailed testing in mammals.

Like calcium it can be delivered into the body as an ion, but like iron or zinc it can also be delivered in a chelated form—that is incorporated into a soluble molecule with a strong complex formation such as EDTA (ethylene diamine tetraacetic acid—molar mass 292), DTPA (diethylene triamine pentaacetic acid—molar mass 393) or GLDA (L-Glutamic diacetic acid, molar mass 351). Alternately it can be delivered incorporated into a smaller chelating agent with low strength complex formation such as NTA (nitrilotriacetic acid, molar mass 191). It can also be delivered incorporated into a variety of other compounds. Delivery may be topical, ingested or injected.

By introducing a trivalent such as scandium, or a higher affinity divalent cation, where no significant toxicity results, it becomes possible to optimize and improve fidelity and processivity of nucleic acid polymerase and reverse transcriptase enzymes.

To the extent that cancers emerge from errors in DNA replication and to the extent that death and declines in health/vitality emerge from poor telomere maintenance, the use of replacement cations—such as in the form of NTA-scandium—can decrease cancer formation rates and slow the rate of cellular senescence.

Telomerase and telomerase RNA are present in somatic cells wherein genes that encode them are suppressed. To this extent, their very low rate of function reduces their effectiveness. There is variation in telomerase function in human populations that relates to longevity (see, for instance, Atzmon, G, et al. Genetic variation in human telomerase is associated with telomere length in Ashkenazi centenarians. 106 PNAS 1710-1717 (2010)).

The function of reverse transcription in telomerase is far different from the function of reverse transcriptase in viral replication. All that is required is for the telomerase is to form a short repetitive strip of single strand DNA with a repeat pattern of TTAGGG. However, the telomerase RNA has a number of highly conserved areas of secondary structure that appear to play a role in the complex process of determining when to trigger telomerase activity, carrying out the generation of the tandem repeats of TTAGGG, and then repositioning the enzyme to repetitively extend the replacement telomere strand.

The increased adhesion provided by trivalent cations such as Scandium can affect various functions in the telomerase transcription from the telomerase gene, formation of the telomere RNA, assembly of the telomerase ribonucleoprotein, and the processivity of the reverse transcription process. These effects can cause an increase in telomere length with a resultant delay in cellular senescence. These changes can result in improved longevity.

What is claimed is:

1. A pharmaceutical agent comprising
a) a trivalent cation from the group of Scandium, Yttrium, or any of the lanthanide series of elements (Lanthanum, Cerium, Praesodymium, Neodymium, Prometheum, Samarium, Europium, Gadolinium, Terbium, Dysprosium, Holmium, Erbium, Thulium, Ytterbium or Lutetium),
b) wherein such trivalent cation is administered for the purpose of associating it with telomerase enzymes in the body.

2. A pharmaceutical agent comprising,
a) the trivalent cation Scandium,
b) wherein such trivalent cation is administered for the purpose of associating it with telomerase enzymes in the body.

3. A food additive agent comprising
a) a trivalent cation from the group of Scandium, Yttrium, or any of the lanthanide series of elements (Lanthanum, Cerium, Praesodymium, Neodymium, Prometheum, Samarium, Europium, Gadolinium, Terbium, Dysprosium, Holmium, Erbium, Thulium, Ytterbium or Lutetium),
b) wherein such trivalent cation is administered for the purpose of associating it with telomerase enzymes in the body.

4. A food additive agent comprising
a) the trivalent cation Scandium,
b) wherein such trivalent cation is administered for the purpose of associating it with telomerase enzymes in the body.

5. A topical agent comprising
a) a trivalent cation from the group of Scandium, Yttrium, or any of the lanthanide series of elements (Lanthanum, Cerium, Praesodymium, Neodymium, Prometheum, Samarium, Europium, Gadolinium, Terbium, Dysprosium, Holmium, Erbium, Thulium, Ytterbium or Lutetium),
b) wherein such trivalent cation is administered for the purpose of associating it with telomerase enzymes in the body.

6. A topical agent comprising
a) the trivalent cation Scandium,
b) wherein such trivalent cation is administered for the purpose of associating it with telomerase enzymes in the body.

7. A dietary supplement agent comprising
a) a trivalent cation from the group of Scandium, Yttrium, or any of the lanthanide series of elements (Lanthanum, Cerium, Praesodymium, Neodymium, Prometheum, Samarium, Europium, Gadolinium, Terbium, Dysprosium, Holmium, Erbium, Thulium, Ytterbium or Lutetium),
b) wherein such trivalent cation is administered for the purpose of associating it with telomerase enzymes in the body.

8. A dietary supplement agent comprising
a) the trivalent cation Scandium,
b) wherein such trivalent cation is administered for the purpose of associating it with telomerase enzymes in the body.

9. The agents of claim 1, 2, 3, 4, 5, 6, 7, or 8, wherein the trivalent cation is incorporated into a chelating molecule.

10. The agents of claim 1, 2, 3, 4, 5, 6, 7, or 8, wherein the trivalent cation is incorporated into a chelating molecule wherein that molecule is EDTA (ethylenediamine tetraacetic acid).

11. The agents of claim 1, 2, 3, 4, 5, 6, 7, or 8, wherein the trivalent cation is incorporated into a chelating molecule wherein that molecule is DTPA (diethylenetriamine pentaacetic acid).

12. The agents of claim 1, 2, 3, 4, 5, 6, 7, or 8, wherein the trivalent cation is incorporated into a chelating molecule wherein that molecule is NTA (nitrilotriacetic acid).

13. The agents of claim 1, 2, 3, 4, 5, 6, 7, or 8, wherein the trivalent cation is incorporated into a chelating molecule wherein that molecule is gluconate.

14. A method of increasing the processivity of telomerase enzymes in the mammalian body by the administration of an agent comprising a trivalent cation.

15. A method of increasing the processivity of telomerase enzymes in the human body by the administration of an agent comprising a trivalent cation.

16. A method of increasing the fidelity of telomerase enzymes in the mammalian body by the administration of an agent comprising a trivalent cation.

17. A method of increasing the fidelity of telomerase enzymes in the human body by the administration of an agent comprising a trivalent cation.

18. A method of increasing the number of types of cell which exhibit effective activity of telomerase enzymes in the mammalian body by the administration of an agent comprising a trivalent cation.

19. A method of increasing the number of types of cell which exhibit effective activity of telomerase enzymes in the human body by the administration of an agent comprising a trivalent cation.

20. A method of increasing the number of types of cell which exhibit effective activity of telomerase enzymes in the mammalian body by the administration of an agent comprising a trivalent cation.

21. A method of increasing the number of types of cell which exhibit effective activity of telomerase enzymes in the human body by the administration of an agent comprising a trivalent cation.

22. A method of delaying the onset of cellular senescence for any type of cell by increasing the functionality of telomerase enzymes in the mammalian body by the administration of an agent comprising a trivalent cation.

23. A method of delaying the onset of cellular senescence for any type of cell by increasing the functionality of telomerase enzymes in the human body by the administration of an agent comprising a trivalent cation.

24. A method of increasing the longevity of a mammal by changing the function of telomerase enzymes by, the administration of an agent comprising a trivalent cation.

25. A method of increasing the longevity of a human by changing the function of telomerase enzymes by the administration of an agent comprising a trivalent cation
